SEQUENCE LISTING (21 sequences)

SEQ ID NO: 1 - protein, 121 aa, synthetic construct
  REGION 1..121: note = Description of Artificial Sequence: Heavy Chain Variable Region - VH
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 2 - protein, 111 aa, synthetic construct
  REGION 1..111: note = Description of Artificial Sequence: Light Chain Variable Region - VL
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 3 - protein, 5 aa, synthetic construct
  REGION 1..5: note = Description of Artificial Sequence: Heavy Chain Variable Region CDR1
SSWMN

SEQ ID NO: 4 - protein, 17 aa, synthetic construct
  REGION 1..17: note = Description of Artificial Sequence: Heavy Chain Variable Region CDR2
RIYPGDGDTNYNVKFKG

SEQ ID NO: 5 - protein, 12 aa, synthetic construct
  REGION 1..12: note = Description of Artificial Sequence: Heavy Chain Variable Region CDR3
SGFITTVRDFDY

SEQ ID NO: 6 - protein, 15 aa, synthetic construct
  REGION 1..15: note = Description of Artificial Sequence: Light Chain Variable Region CDR1
RASESVDTFGISFMN

SEQ ID NO: 7 - protein, 7 aa, synthetic construct
  REGION 1..7: note = Description of Artificial Sequence: Light Chain Variable Region CDR2
EASNQGS

SEQ ID NO: 8 - protein, 9 aa, synthetic construct
  REGION 1..9: note = Description of Artificial Sequence: Light Chain Variable Region CDR3
QQSKEVPFT

SEQ ID NO: 9 - protein, 121 aa, synthetic construct
  REGION 1..121: note = Description of Artificial Sequence: Heavy Chain Variable Region - VH
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 10 - protein, 112 aa, synthetic construct
  REGION 1..112: note = Description of Artificial Sequence: Light Chain Variable Region - VL
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 11 - protein, 5 aa, synthetic construct
  REGION 1..5: note = Description of Artificial Sequence: Heavy Chain Variable Region CDR1
SYVMH

SEQ ID NO: 12 - protein, 6 aa, synthetic construct
  REGION 1..6: note = Description of Artificial Sequence: Heavy Chain Variable Region CDR2
NPYNDG

SEQ ID NO: 13 - protein, 12 aa, synthetic construct
  REGION 1..12: note = Description of Artificial Sequence: Heavy Chain Variable Region CDR3
GTYYYGTRVFDY

SEQ ID NO: 14 - protein, 16 aa, synthetic construct
  REGION 1..16: note = Description of Artificial Sequence: Light Chain Variable Region CDR1
RSSKSLQNVNGNTYLY

SEQ ID NO: 15 - protein, 7 aa, synthetic construct
  REGION 1..7: note = Description of Artificial Sequence: Light Chain Variable Region CDR2
RMSNLNS

SEQ ID NO: 16 - protein, 9 aa, synthetic construct
  REGION 1..9: note = Description of Artificial Sequence: Light Chain Variable Region CDR3
MQHLEYPIT

SEQ ID NO: 17 - DNA, 145 bp, synthetic construct
  misc_feature 1..145: note = Description of Artificial Sequence: AAV2 ITR
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 18 - DNA, 2211 bp, synthetic construct
  misc_feature 1..2211: note = Description of Artificial Sequence: AAV9 Nucleic Acid Sequence
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 19 - protein, 736 aa, synthetic construct
  REGION 1..736: note = Description of Artificial Sequence: AAV9 Amino Acid Sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - protein, 310 aa, synthetic construct
  REGION 1..310: note = Description of Artificial Sequence: Streptococcus pyogenes IdeS
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 21 - protein, 349 aa, synthetic construct
  REGION 1..349: note = Description of Artificial Sequence: Streptococcus equi IdeZ
MKTIAYPNKPHSLSAGLLTAIAIFSLASSNITYADDYQRNATEAYAKEVPHQITSVWSKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

(19) United States
(12) Patent Application Publication
(10) Pub. No.: US 2026/0130990 A1
(43) Pub. Date: May 14, 2026

(54) METHODS FOR IMPROVED THERAPEUTIC USE OF RECOMBINANT AAV

(71) Applicant: Ultragenyx Pharmaceutical Inc., Novato, CA (US)

(72) Inventors: Barbara Anne Sullivan, Tiburon, CA (US); Kathleen Phillips McKeever, San Anselmo, CA (US)

(21) Appl. No.: 19/442,866

(22) Filed: Jan. 7, 2026

Related U.S. Application Data
(63) Continuation of application No. 17/764,295, now Pat. No. 12,544,437.
(60) Provisional application No. 62/910,790, filed on Oct. 4, 2019.

(51) Int. Cl. A61K 39/395; A61K 31/5377; A61K 31/573; A61K 31/675; A61K 31/69; A61K 38/18; A61K 38/36; A61K 38/37; A61K 38/44; A61K 38/45; A61K 38/46; A61K 38/48; A61K 38/53; A61K 48/00

(52) U.S. Cl. CPC A61K 39/3955; A61K 31/5377; A61K 31/573; A61K 31/675; A61K 31/69; A61K 38/1825; A61K 38/36; A61K 38/37; A61K 38/44; A61K 38/45; A61K 38/46; A61K 38/465; A61K 38/48; A61K 38/4873; A61K 38/53; A61K 48/0083

(57) ABSTRACT

Provided herein are methods for managing host immune responses to improve therapeutic outcomes in adeno-associated virus (AAV)-mediated gene therapy. Such methods may include administering a recombinant adeno-associated virus (rAAV) to a subject following administration of a CD19 inhibitor, e.g., an anti-CD19 antibody. The methods described herein can facilitate improved transgene expression, help overcome pre-existing NAbs, and/or permit redosing with the same or substantially similar rAAV or transgene.

### CROSS-REFERENCE TO RELATED APPLICATIONS

This application is a continuation of U.S. application Ser. No. 17/764,295, filed on Mar. 28, 2022, which is a U.S. national stage application, filed under 35 U.S.C. § 371, of International Application No. PCT/US2020/053805, filed on Oct. 1, 2020, which claims the benefit of and priority to U.S. Provisional Application No. 62/910,790, filed on Oct. 4, 2019, the entire disclosure of each of which are hereby incorporated by reference herein in its entirety for all purposes.

### SEQUENCE LISTING

The instant application contains a Sequence Listing which has been submitted electronically in XML format and is hereby incorporated by reference in its entirety. Said XML copy, created on Jan. 7, 2026, is named “00021.016.1401_SL.xml” and is 32,280 bytes in size.

### TECHNICAL FIELD OF THE INVENTION

The present disclosure relates generally to methods for managing host immune responses to improve therapeutic outcomes in adeno-associated virus (AAV)-mediated gene therapy.

### BACKGROUND OF THE INVENTION

A major challenge to the successful application of AAV-mediated gene therapy is the induction of innate and adaptive immune responses to the AAV capsid, vector DNA, and transgene product (See Corti et al., 2014, Mol Ther Methods Clin Dev 1, 14033). The innate immune response to a gene therapy product (e.g., a recombinant AAV, i.e., rAAV) may eliminate the product and transfected cells, decreasing both the intensity and duration of transgene expression (See Bessis et al., 2004, Gene Therapy 11, S10-S17). The adaptive immune response develops later, and plays a crucial role—it includes a humoral response characterized by the production of neutralizing antibodies (NAbs) specific to the capsid or an expressed transgene and a cell-mediated response involving T cells and NK cells (See id.). Adaptive immunity not only contributes to eliminating the AAV product and transfected cells from the body, but also results in a memory response that impedes the delivery of the same rAAV or transgene (See id.). Lastly, some individuals may be exposed naturally to AAV early in life, leading to the induction of AAV NAbs, which can prohibit the use of AAV-based gene therapy in these patients.

For the foregoing reasons, there is a need to manage the host immune response to facilitate improved transgene expression, to overcome pre-existing NAbs, and/or to permit redosing with the same rAAV or transgene. The present invention addresses this need via the pharmacological modulation of the humoral immune response using a CD19 inhibitor, e.g., an anti-CD19 antibody.

### SUMMARY OF THE INVENTION

This invention provides methods for managing host immune responses to improve therapeutic outcomes in AAV gene therapy. More specifically, provided herein are methods comprising the administration of a CD19 inhibitor prior to the administration of an AAV gene therapy product.

In a first aspect, the present disclosure provides a method for administering a recombinant adeno-associated virus (rAAV) to a subject, wherein said method comprises first administering a CD19 inhibitor to said subject and thereafter administering the rAAV to said subject.

In one embodiment, the CD19 inhibitor is administered to the subject at least about 12 hours before administration of the rAAV. In another embodiment, the CD19 inhibitor is administered to the subject at least about 24 hours before administration of the rAAV. In yet another embodiment, the CD19 inhibitor is administered to the subject at least about 2 days before administration of the rAAV. In yet another embodiment, the CD19 inhibitor is administered to the subject at least about 3, 4, 5, 6, 7, or more days before administration of the rAAV. In yet another embodiment, the CD19 inhibitor is administered to the subject at least about 7, 14, 21, or more days before administration of the rAAV. In an exemplary embodiment, the CD19 inhibitor is administered to the subject at or about 7 days before administration of the rAAV.

In one embodiment, the CD19 inhibitor is administered once before administration of the rAAV. In another embodiment, the CD19 inhibitor is administered twice before administration of the rAAV. In yet another embodiment, the CD19 inhibitor is administered 3, 4, 5, or more times before administration of the rAAV. In an exemplary embodiment, the CD19 inhibitor is administered once before administration of the rAAV.

In one embodiment, the CD19 inhibitor is selected from an anti-CD19 antibody, an anti-CD19 single-chain variable fragment (scFv), a CD19 antisense oligonucleotide, a CD19 small interfering RNA (siRNA), and a small molecule inhibitor of CD19. In an exemplary embodiment, the CD19 inhibitor is an anti-CD19 antibody.

In various embodiments described herein, the CD19 inhibitor may be an anti-CD19 antibody. In some embodiments, the anti-CD19 antibody may be selected from inebilizumab, tafasitamab, and FMC63. In an exemplary embodiment, the anti-CD19 antibody is inebilizumab.

In certain embodiments where the CD19 inhibitor is an anti-CD19 antibody, the anti-CD19 antibody may be administered at a dose of about 10 mg to about 3000 mg. In some embodiments, the anti-CD19 antibody may be administered at a dose of about 50 mg to about 2000 mg. In some embodiments, the anti-CD19 antibody may be administered at a dose of about 100 mg to about 1000 mg. In some embodiments, the anti-CD19 antibody may be administered at a dose of about 200 mg to about 500 mg. In some embodiments, the anti-CD19 antibody may be administered at a dose of about 250 mg to about 350 mg. In an exemplary embodiment, the anti-CD19 antibody may be administered at a dose of about 300 mg.

In certain embodiments where the CD19 inhibitor is an anti-CD19 antibody, the anti-CD19 antibody may be administered at a dose of about 100 mg, about 200 mg, about 300 mg, about 400 mg, about 500 mg, about 600 mg, about 700 mg, about 800 mg, about 900 mg, or about 1000 mg. In an exemplary embodiment, the anti-CD19 antibody is inebilizumab and is administered at a dose of 300 mg.

In certain embodiments where the CD19 inhibitor is an anti-CD19 antibody, the anti-CD19 antibody may be administered at a dose of about 0.1 mg/kg to about 50 mg/kg. In some embodiments, the anti-CD19 antibody may be administered at a dose of about 1 mg/kg to about 10 mg/kg. In some embodiments, the anti-CD19 antibody may be administered at a dose of about 2 mg/kg to about 8 mg/kg. In some embodiments, the anti-CD19 antibody may be administered at a dose of about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, or about 8 mg/kg.

In certain embodiments where the CD19 inhibitor is an anti-CD19 antibody, the anti-CD19 antibody may formulated in a pharmaceutical composition that comprises the anti-CD19 antibody and a pharmaceutically acceptable carrier or excipient. In some embodiments, the pharmaceutical composition is formulated for subcutaneous, intramuscular, intradermal, intraperitoneal, or intravenous administration. In an exemplary embodiment, the pharmaceutical composition is formulated for intravenous administration.

In some embodiments, the CD19 inhibitor is administered subcutaneously, intramuscularly, intradermally, intraperitoneally, or intravenously. In an exemplary embodiment, the CD19 inhibitor is administered intravenously.

In some embodiments, the method for administering a recombinant adeno-associated virus (rAAV) to a subject may further comprise the administration of intravenous immune globulin (IVIg) before, simultaneous with, or after administration of the CD19 inhibitor. In some embodiments, the CD19 inhibitor and the IVIg may be administered simultaneously. In some embodiments, the CD19 inhibitor and the IVIg may be administered sequentially. In some embodiments, the CD19 inhibitor and the IVIg may be administered separately.

In some embodiments, the method for administering a recombinant adeno-associated virus (rAAV) to a subject may further comprise the administration of a corticosteroid before, simultaneous with, or after administration of the CD19 inhibitor. In some embodiments, the CD19 inhibitor and the corticosteroid may be administered simultaneously. In some embodiments, the CD19 inhibitor and the corticosteroid may be administered sequentially. In some embodiments, the CD19 inhibitor and the corticosteroid may be administered separately. In some embodiments, the corticosteroid is selected from prednisolone, dexamethasone, hydrocortisone, methylprednisolone, betamethasone, cortisone, prednisone, budesonide, and triamcinolone. In some embodiments, the corticosteroid is prednisolone. In some embodiments, the corticosteroid is administered at 60 mg per day. In some embodiments, prednisolone is administered at about 5 days prior to administration of the rAAV. In some embodiments, the corticosteroid is administered at 60 mg per day for 4 weeks with a first dose occurring 5 days prior to administration of the rAAV.

In some embodiments, the method for administering a recombinant adeno-associated virus (rAAV) to a subject may further comprise the administration of an agent before, simultaneous with, or after administration of the CD19 inhibitor. In some embodiments, the CD19 inhibitor and the agent may be administered simultaneously. In some embodiments, the CD19 inhibitor and the agent may be administered sequentially. In some embodiments, the CD19 inhibitor and the agent may be administered separately. In some embodiments, the agent is selected from a proteasome inhibitor, a TLR antagonist, an immunosuppressive macrocycle, an antimetabolite, a cGAS-STING antagonist, IL-2, an IL-2 mutein, an anti-CD38 antibody, an immunomodulatory drug (IMiD), an anti-B-cell maturation antigen (BCMA) agent, an anti-SLAM family member 7 (SLAMF7) antibody, a mammalian target of rapamycin (mTOR) inhibitor, SEL-212, cyclophosphamide, mycophenolate mofetil, a phosphoinositide 3-kinase inhibitor, a Bruton's tyrosine kinase inhibitor, a sphingosine-1-phosphate receptor modulator, an anti-B-cell activating factor (BAFF or also known as tumor necrosis factor ligand superfamily member 13B) inhibitor, or an IgG-degrading protease. In some embodiments, the proteasome inhibitor is selected from bortezomib, carfilzomib, ixazomib, oprozomib, delanzomib, and marizomib. In an exemplary embodiment, the proteasome inhibitor is bortezomib. In some embodiments, the IgG-degrading protease is IdeS of Streptococcus pyogenes or an engineered variant thereof. In some embodiments, the IgG-degrading protease is IdeZ of Streptococcus equi or an engineered variant thereof.

In some embodiments, the recombinant adeno-associated virus (rAAV) administered according to a method of the application comprises an AAV capsid and a vector genome packaged therein.

In some embodiments, the AAV capsid is from an AAV of serotype 1, 2, 3, 4, 5, 6, 7, 8, 9, 10, 11, 12, rh10, hu37 (i.e., AAV1, AAV2, AAV3, AAV4, AAV5, AAV6, AAV7, AAV9, AAV10, AAV11, AAV12, AAVrh10, AAVhu37), or an engineered variant thereof. In an exemplary embodiment, the AAV capsid is an AAV serotype 9 (AAV9) capsid, an AAV9 variant capsid, an AAV serotype 8 (AAV8) capsid, an AAV8 variant capsid, an AAV serotype 2 (AAV2) capsid, or an AAV serotype hu37 (AAVhu37) capsid.

In some embodiments, the vector genome packaged in the rAAV comprises a coding sequence for a protein transgene. In one embodiment, the coding sequence is a native coding sequence. In another embodiment, the coding sequence is a codon-optimized coding sequence. In some embodiments, coding sequence expresses a protein transgene selected from ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit α (PCCA) and propionyl-CoA carboxylase subunit β (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, and a dystrophin gene product (e.g., a minidystrophin or microdystrophin).

In some embodiments, the rAAV is administered subcutaneously, intramuscularly, intradermally, intraperitoneally, intrathecally, intracerebroventricularly, or intravenously. In an exemplary embodiment, the rAAV is administered intravenously. In some embodiments, the rAAV is administered at a dose of about 1×10¹¹ to about 1×10¹⁴ genome copies (GC)/kg. In further embodiments, the rAAV is administered at a dose of about 1×10¹² to about 1×10¹³ genome copies (GC)/kg. In some embodiments, a single dose of rAAV is administered. In other embodiments, multiple doses of rAAV are administered.

In some embodiments, the subject is a human. In one embodiment, the human subject is an adult subject, i.e., a human subject greater than 18 years old. In one embodiment, the human subject is a pediatric subject, i.e., a human subject of ages 0-18 years old inclusive.

In a second aspect, the present disclosure provides a method for administering at least two doses of recombinant adeno-associated virus (rAAV) to a subject, wherein said method comprises:
(a) administering a first dose of a CD19 inhibitor to said subject and thereafter administering a first rAAV to said subject, and
(b) administering a second dose of a CD19 inhibitor to said subject and thereafter administering a second rAAV to said subject.

In some embodiments according to this second aspect, the CD19 inhibitor of the first and second doses is the same CD19 inhibitor. Alternatively, the CD19 inhibitor of the first dose may be a different CD19 inhibitor relative to the second dose.

In some embodiments according to this second aspect, the first rAAV and the second rAAV are the same rAAV. Alternatively, in some embodiments, the first rAAV and the second rAAV are different.

In some embodiments according to this second aspect, the first rAAV and the second rAAV contain the same vector capsid. In an alternative embodiment, the first rAAV and the second rAAV contain different vector capsids.

In some embodiments according to this second aspect, the first rAAV and the second rAAV express the same transgene. In an alternative embodiment, the first rAAV and the second rAAV express different transgenes.

In some embodiments according to this second aspect, administration of the first dose of the CD19 inhibitor occurs more than 12 months prior to administration of the second dose of the CD19 inhibitor. In one embodiment, administration of the first dose of the CD19 inhibitor occurs more than 2 years prior to administration of the second dose of the CD19 inhibitor. In another embodiment, administration of the first dose of the CD19 inhibitor occurs more than 3, 4, 5, 6, 7, 8, 9, 10, 15, or 20 years prior to administration of the second dose of the CD19 inhibitor.

In some embodiments according to this second aspect, the CD19 inhibitor of the first dose is an anti-CD19 antibody. In some embodiments according to this second aspect, the CD19 inhibitor of the second dose is an anti-CD19 antibody. In some embodiments according to this second aspect, the CD19 inhibitor of the first and second doses is an anti-CD19 antibody. In one embodiment, the anti-CD19 antibody is selected from inebilizumab, tafasitamab, and FMC63. In an exemplary embodiment, the anti-CD19 antibody is inebilizumab.

In some embodiments according to this second aspect, the amount of CD19 inhibitor administered in the first and second doses is the same. Alternatively, the amount of CD19 inhibitor administered in the first dose may be different than the amount of the CD19 inhibitor administered in the second dose. In an exemplary embodiment, the amount of CD19 inhibitor administered in the first and second doses is the same. In another exemplary embodiment, the CD19 inhibitor is inebilizumab and the first dose is 300 mg and the second dose is 300 mg.

These and other aspects and features of the invention are described in the following sections of the application.

### DETAILED DESCRIPTION OF THE INVENTION

This invention provides methods for managing host immune responses to improve therapeutic outcomes in adeno-associated virus (AAV)-mediated gene therapy. In some embodiments, the methods described herein may facilitate improved transgene expression, help overcome pre-existing NAbs, and/or permit redosing with the same or substantially similar rAAV or transgene. In some embodiments, the methods provided by this disclosure include administering a recombinant adeno-associated virus (rAAV) to a subject following administration of a CD19 inhibitor, e.g., an anti-CD19 antibody. Such methods can be useful for the prevention, treatment, or amelioration of various diseases and disorders as further described herein.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 12 recognized serotypes of AAV (AAV1-12).

Administration/Administer: To provide or give a subject an agent, such as a therapeutic agent (e.g., a recombinant AAV or a CD19 inhibitor), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Antibody: The terms “antibody” or “antibodies” encompass monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity, disulfide-linked Fvs (sdFv), and anti-idiotypic (anti-Id) antibodies (including, e.g., anti-Id antibodies to antibodies used in any method of the present disclosure), intrabodies, and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgAQ1 and IgA2) or subclass.

Antibody-dependent cell-mediated cytotoxicity: “Antibody-dependent cell-mediated cytotoxicity” and “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell.

CD19 Inhibitor: A “CD19 inhibitor” refers to an agent that either (a) depletes (i.e., diminishes in number or quantity) cells expressing CD19, e.g., B cells or plasma cells; or (b) directly or indirectly decreases, blocks, inhibits, abrogates, or interferes with signal transduction resulting from the interaction of CD19 with one or more of its binding partners. Such agents include, but are not limited to, anti-CD19 antibodies, anti-CD19 scFvs, CD19 antisense oligonucleotides, CD19 siRNAs, and small molecule inhibitors of CD19.

Coding Sequence: A “coding sequence” means the nucleotide sequence encoding a polypeptide in vitro or in vivo when operably linked to appropriate regulatory sequences. The coding sequence may or may not include regions preceding and following the coding region, e.g., 5′ untranslated (5′ UTR) and 3′ untranslated (3′ UTR) sequences, as well as intervening sequences (introns) between individual coding segments (exons).

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Complement dependent cytotoxicity: “Complement dependent cytotoxicity” and “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen.

Enhancer: A nucleic acid sequence that increases the rate of transcription by increasing the activity of a promoter.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus, or cell) has been substantially separated or purified away from other biological components in the cell or tissue of the organism, or the organism itself, in which the component naturally occurs, such as other chromosomal and extra-chromosomal DNA and RNA, proteins, and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g., a gene). A promoter includes necessary nucleic acid sequences near the start site of transcription. Many promoter sequences are known to the person skilled in the art and even a combination of different promoter sequences in artificial nucleic acid molecules is possible. As used herein, gene-specific endogenous promoter refers to native promoter element that regulates expression of the endogenous gene of interest.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins, and viruses that have been altered solely by addition, substitution, or deletion of a portion of a natural nucleic acid molecule, protein, or virus. As used herein, “recombinant AAV” refers to an AAV particle in which a recombinant nucleic acid molecule such as a recombinant nucleic acid molecule encoding a transgene has been packaged.

Serotype: A group of closely related microorganisms (such as viruses) distinguished by a characteristic set of antigens.

Stuffer sequence: Refers to a sequence of nucleotides contained within a larger nucleic acid molecule (such as a vector) that is typically used to create desired spacing between two nucleic acid features (such as between a promoter and a coding sequence), or to extend a nucleic acid molecule so that it is of a desired length. Stuffer sequences do not contain protein coding information and can be of unknown/synthetic origin and/or unrelated to other nucleic acid sequences within a larger nucleic acid molecule.

Subject: Living multi-cellular vertebrate organisms, a category that includes human and non-human mammals. In some embodiments, the subject is a human. In one embodiment, the human subject is an adult subject, i.e., a human subject greater than 18 years old. In one embodiment, the human subject is a pediatric subject, i.e., a human subject of ages 0-18 years old inclusive.

Synthetic: Produced by artificial means in a laboratory, for example a synthetic nucleic acid can be chemically synthesized in a laboratory.

Therapeutically effective amount: A quantity of a specified pharmaceutical or therapeutic agent (e.g., a recombinant AAV or a CD19 inhibitor) sufficient to achieve a desired effect in a subject, or in a cell, being treated with the agent. The effective amount of the agent will be dependent on several factors, including, but not limited to the subject or cells being treated, and the manner of administration of the therapeutic composition.

Transgene: A “transgene” refers to a polynucleotide that is introduced into a cell and is capable of being transcribed into RNA and optionally translated and/or expressed under appropriate conditions. The term “transgene” may also be used in reference to the expressed polypeptide, i.e., a protein transgene. The transgene may confer a desired property to a cell into which it was introduced, or otherwise lead to a desired therapeutic outcome. In some instances, the transgene may be transcribed and translated to provide a functionally active protein transgene to a subject suffering from a disorder associated with a deficiency of active protein. Any number of protein transgenes may be delivered in conjunction with the present invention including, but not limited to, ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit α (PCCA) and propionyl-CoA carboxylase subunit β (PCCB). In some embodiments, one or more protein transgenes that are delivered in conjunction with the present invention are a variant of any of the protein transgenes including, but not limited to, a variant of ornithine transcarbamylase (OTC), a variant of glucose 6-phosphatase (G6Pase), a variant of factor VIII, a variant of factor IX, a variant of ATP7B, a variant of phenylalanine hydroxylase (PAH), a variant of argininosuccinate synthetase, a variant of cyclin-dependent kinase-like 5 (CDKL5), a variant of propionyl-CoA carboxylase subunit α (PCCA) and a variant of propionyl-CoA carboxylase subunit β (PCCB).

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

This invention provides methods for managing host immune responses to improve therapeutic outcomes in AAV gene therapy. More specifically, provided herein are methods comprising the administration of an rAAV gene therapy product following administration of an inhibitor of CD19. Such methods can be useful in suppressing the development of long-lasting humoral immunity that may otherwise be generated against an rAAV gene therapy product or the expressed transgene. As described herein, methods utilizing inhibitors of CD19 represent an improvement over current approaches that employ anti-CD20 antibodies, such as those being employed in Clinical Trial No. NCT02240407 (“Re-administration of Intramuscular AAV9 in Patients with Late-Onset Pompe Disease (AAV9-GAA_IM)”) and Clinical Trial No. NCT02525172 (“Immune Modulation Therapy for Pompe Disease”).

Although the mechanisms underlying the maintenance of long-lasting humoral immunity are not well understood, it is well established that antigen-specific antibody responses can last as long as a lifetime (See Amanna et al., 2007, NEJM 357 (19): 1903-15 and Crotty et al., 2003, The Journal of Immunology, 171:4969-4973). Upon encountering an antigen, a subject's B cells proliferate and give rise to memory B cells and clonally related plasma cells, with memory B cells giving rise to additional plasma cells upon antigen reencounter. Interestingly, B-cell depletion studies using anti-CD20 antibodies suggest that at least some plasma cells are maintained independent of regeneration from B cells (See Bhoj et al., 2016, Blood 12:360-370 and Owczarczyk et al., 2011, Science Translational Medicine, Vol 3 Issue 101, 101ra92). This implies that long-lived humoral immunity may theoretically be maintained by plasma cells and that such plasma cells are not impacted via existing B-cell depletion strategies that employ an anti-CD20 antibody such as rituximab. Accordingly, novel approaches are needed to additionally deplete certain plasma cells that may otherwise prohibit redosing with the same rAAV or an AAV expressing the same or substantially similar protein transgene.

It has been proposed that humoral immunity relies on distinct subsets of plasma cells with complementary functions and different dynamics (See Mei et al., 2015, Blood 125:1739-1748). Recent studies have shown that some long-lived plasma cells in human bone marrow express CD19 and that these plasma cells can be present decades after antigen encounter (See Brynjolfsson et al., 2017, Blood Adv. 1:835-838). Human CD19 is a 95 kilodalton transmembrane glycoprotein belonging to the immunoglobulin (lg) superfamily. CD19 is classified as a type I transmembrane protein, with a single transmembrane domain, a cytoplasmic C-terminus, and an extracellular N-terminus. CD19 is specifically expressed in normal and neoplastic B cells, as well as follicular dendritic cells. During B cell lymphopoiesis, the surface expression of CD19 begins during immunoglobulin gene rearrangement. The surface density of CD19 is highly regulated throughout B cell development and maturation, until the loss of expression during terminal plasma cell differentiation. Given the expression of CD19 on B cells as well as plasma cells, it can represent as an improved target over CD20.

In a first aspect, the present disclosure provides a method for administering a recombinant adeno-associated virus (rAAV) to a subject, wherein said method comprises first administering a CD19 inhibitor to said subject and thereafter administering the rAAV to said subject.

In one embodiment, the CD19 inhibitor is selected from an anti-CD19 antibody, an anti-CD19 scFv, a CD19 antisense oligonucleotide, a CD19 siRNA, and a small molecule inhibitor of CD19.

In one embodiment, the CD19 inhibitor is an anti-CD19 antibody. In some embodiments, the anti-CD19 antibody may be selected from inebilizumab, tafasitamab, and FMC63.

In one embodiment, the anti-CD19 antibody is inebilizumab, which is described in U.S. Pat. Nos. 8,323,653, 8,883,992, and 9,896,505, the disclosures of which are herein incorporated by reference.

In one embodiment, the anti-CD19 antibody comprises a heavy chain variable region (VH) comprising the amino acid sequence of SEQ ID NO: 1. In one embodiment, the anti-CD19 antibody comprises a light chain variable region (VL) comprising the amino acid sequence of SEQ ID NO: 2. In one embodiment, the anti-CD19 antibody comprises a heavy chain variable region (VH) comprising the amino acid sequence of SEQ ID NO: 1 and a light chain variable region (VL) comprising the amino acid sequence of SEQ ID NO: 2.

In one embodiment, the anti-CD19 antibody comprises a heavy chain variable region (VH) comprising at least one CDR having an amino acid sequence selected from SEQ ID NO: 3, SEQ ID NO: 4, and SEQ ID NO: 5. In one embodiment, the anti-CD19 antibody comprises a light chain variable region (VL) comprising at least one CDR having an amino acid sequence selected from SEQ ID NO: 6, SEQ ID NO: 7, and SEQ ID NO: 8. In one embodiment, the anti-CD19 antibody comprises a heavy chain variable region (VH) comprising at least one CDR having an amino acid sequence selected from SEQ ID NO: 3, SEQ ID NO: 4, and SEQ ID NO: 5 and a light chain variable region (VL) comprising at least one CDR having an amino acid sequence selected from SEQ ID NO: 6, SEQ ID NO: 7, and SEQ ID NO: 8.

In one embodiment, the anti-CD19 antibody comprises a heavy chain variable region (VH) comprising CDRs having amino acid sequences of SEQ ID NO: 3 (CDR1), SEQ ID NO: 4 (CDR2), and SEQ ID NO: 5 (CDR3). In one embodiment, the anti-CD19 antibody comprises a light chain variable region (VL) comprising CDRs having amino acid sequences of SEQ ID NO: 6 (CDR1), SEQ ID NO: 7 (CDR2), and SEQ ID NO: 8 (CDR3). In one embodiment, the anti-CD19 antibody comprises a heavy chain variable region (VH) comprising CDRs having amino acid sequences of SEQ ID NO: 3 (CDR1), SEQ ID NO: 4 (CDR2), and SEQ ID NO: 5 (CDR3) and a light chain variable region (VL) comprising CDRs having amino acid sequences of SEQ ID NO: 6 (CDR1), SEQ ID NO: 7 (CDR2), and SEQ ID NO: 8 (CDR3).

In one embodiment, the anti-CD19 antibody is tafasitamab, which is described in U.S. Pat. Nos. 8,524,867, 9,803,020, and WO/2018/002031, the disclosures of which are herein incorporated by reference.

In one embodiment, the anti-CD19 antibody comprises a heavy chain variable region (VH) comprising the amino acid sequence of SEQ ID NO: 9. In one embodiment, the anti-CD19 antibody comprises a light chain variable region (VL) comprising the amino acid sequence of SEQ ID NO: 10. In one embodiment, the anti-CD19 antibody comprises a heavy chain variable region (VH) comprising the amino acid sequence of SEQ ID NO: 9 and a light chain variable region (VL) comprising the amino acid sequence of SEQ ID NO: 10.

In one embodiment, the anti-CD19 antibody comprises a heavy chain variable region (VH) comprising at least one CDR having an amino acid sequence selected from SEQ ID NO: 11, SEQ ID NO: 12, and SEQ ID NO: 13. In one embodiment, the anti-CD19 antibody comprises a light chain variable region (VL) comprising at least one CDR having an amino acid sequence selected from SEQ ID NO: 14, SEQ ID NO: 15, and SEQ ID NO: 16. In one embodiment, the anti-CD19 antibody comprises a heavy chain variable region (VH) comprising at least one CDR having an amino acid sequence selected from SEQ ID NO: 11, SEQ ID NO: 12, and SEQ ID NO: 13 and a light chain variable region (VL) comprising at least one CDR having an amino acid sequence selected from SEQ ID NO: 14, SEQ ID NO: 15, and SEQ ID NO: 16.

In one embodiment, the anti-CD19 antibody comprises a heavy chain variable region (VH) comprising CDRs having amino acid sequences of SEQ ID NO: 11 (CDR1), SEQ ID NO: 12 (CDR2), and SEQ ID NO: 13 (CDR3). In one embodiment, the anti-CD19 antibody comprises a light chain variable region (VL) comprising CDRs having amino acid sequences of SEQ ID NO: 14 (CDR1), SEQ ID NO: 15 (CDR2), and SEQ ID NO: 16 (CDR3). In one embodiment, the anti-CD19 antibody comprises a heavy chain variable region (VH) comprising CDRs having amino acid sequences of SEQ ID NO: 11 (CDR1), SEQ ID NO: 12 (CDR2), and SEQ ID NO: 13 (CDR3) and a light chain variable region (VL) comprising CDRs having amino acid sequences of SEQ ID NO: 14 (CDR1), SEQ ID NO: 15 (CDR2), and SEQ ID NO: 16 (CDR3).

In some embodiments, the anti-CD19 antibody may be selected from an anti-CD19 antibody described in any of the following references: WO/2005/012493 (Immunomedics, Inc.), WO/2006/089133 (Duke University), WO/2007/002223 (Medarex, Inc.), WO/2007/076950 (Merck Patent Gmbh), WO/2007/082715 (Friedrich-Alexander-Universitat Erlangen-Nurnberg), WO/2008/022152 (Xencor, Inc.), WO/2008/031056 (Medimmune, LLC), WO/2009/052431 (Seattle Genetics, Inc.), WO/2009/054863 (Medarex, Inc.), WO/2010/053716 (Immunomedics, Inc.), WO/2010/095031 (Glenmark Pharmaceuticals S.A.), WO/2010/102276 (Medimmune, LLC), WO/2011/147834 (Roche Glycart AG), WO/2012/010561 (Universite Claude Bernard Lyon), WO/2012/057765 (The Board of Regents of the University of Texas System), WO/2016/033570 (Juno Therapeutics, Inc.), WO/2017/015783 (Innovative Cellular Therapeutics Co., Ltd.), WO/2017/055328 (F. Hoffmann-La Roche AG), WO/2017/066136 (Eureka Therapeutics, Inc.), WO/2018/002031 (Morphosys AG), WO/2018/108106 (Carsgen Theraeputics, Ltd.), WO/2018/126369 (Shanghai Sidansai Biotechnology Co., Ltd.), and WO/2019/057100 (Wuxi Biologics Co., Ltd.), the disclosures of which are herein incorporated by reference.

In some embodiments, the anti-CD19 antibody mediates antigen-dependent cell-mediated cytotoxicity (ADCC) of cells expressing CD19. In some embodiments, the anti-CD19 antibody mediates complement-dependent cell-mediated cytotoxicity (CDC) of cells expressing CD19. In some embodiments, the anti-CD19 antibody mediates apoptosis of cells expressing CD19. In some embodiments, the anti-CD19 antibody inhibits IgM/CpG stimulated B cell proliferation.

In one embodiment, the anti-CD19 antibody, following administration, may deplete circulating B cells, blood B cells, splenic B cells, marginal zone B cells, follicular B cells, peritoneal B cells, and/or bone marrow B cells.

In one embodiment, the anti-CD19 antibody, following administration, may deplete progenitor B cells, early pro-B cells, late pro-B cells, large-pre-B cells, small pre-B cells, immature B cells, mature B cells, antigen stimulated B cells, and/or plasma cells.

In one embodiment, the anti-CD19 antibody, following administration, may deplete B cells in a human subject. In a specific embodiment, an anti-CD19 antibody administered according to the present invention may achieve at least about 20%, at least about 30%, at least about 40%, at least about 50%, at least about 60%, at least about 70%, at least about 80%, at least about 90%, at least about 95%, or about 100% B cell depletion. In another embodiment, the anti-CD19 antibody, following administration, may deplete B cell subsets in a human subject. In a specific embodiment, the anti-CD19 antibody, following administration, may deplete circulating B cells, blood B cells, splenic B cells, marginal zone B cells, follicular B cells, peritoneal B cells, and/or bone marrow B cells. CD19 is present on the surface of B cells at all developmental stages. An anti-CD19 antibody, following administration, may therefore deplete B cells of all developmental stages. In a specific embodiment, the anti-CD19 antibody, following administration, may achieve depletion of progenitor B cells, early pro-B cells, late pro-B cells, large-pre-B cells, small pre-B cells, immature B cells, mature B cells, antigen stimulated B cells, and/or plasma cells. Depletion of B cells may persist for extended periods of time. In one embodiment, B cell depletion by an anti-CD19 antibody administered according to the invention may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, B cell depletion by an anti-CD19 antibody administered according to the invention may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, B cell depletion by an anti-CD19 antibody administered according to the invention may persist for at least 1 month, at least 2 months, at least 3 months, at least 4 months, at least 5 months, at least 6 months, at least 7 months, at least 8 months, at least 9 months, at least 10 months, at least 11 months or at least 12 months.

In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of circulating B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of blood B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of splenic B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of marginal zone B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of follicular B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of peritoneal B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of bone marrow B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of progenitor B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of early pro-B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of late pro-B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of large pre-B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of small pre-B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of immature B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of mature B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of antigen stimulated B cells. In one embodiment, the anti-CD19 antibody, following administration, depletes at least about 20%, at least about 30%, at least about 40%, at least about 50%, at least about 60%, at least about 70%, at least about 80%, at least about 90%, at least about 95%, or about 100% of plasma cells. Depletion of B cells and/or plasma cells may persist for extended periods of time. In one embodiment, B cell depletion and/or plasma cell depletion by an anti-CD19 antibody may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, B cell depletion and/or plasma cell depletion by an anti-CD19 antibody may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, B cell depletion and/or plasma cell depletion by an anti-CD19 antibody may persist for at least 1 month, at least 2 months, at least 3 months, at least 4 months, at least 5 months, at least 6 months, at least 7 months, at least 8 months, at least 9 months, at least 10 months, at least 11 months, or at least 12 months.

In one embodiment, the CD19 inhibitor is administered to the subject at least about 12 hours before administration of the rAAV. In another embodiment, the CD19 inhibitor is administered to the subject at least about 24 hours before administration of the rAAV. In yet another embodiment, the CD19 inhibitor is administered to the subject at least about 2 days before administration of the rAAV. In yet another embodiment, the CD19 inhibitor is administered to the subject at least about 3, 4, 5, 6, 7, or more days before administration of the rAAV. In yet another embodiment, the CD19 inhibitor is administered to the subject at least about 7, 14, 21, or more days before administration of the rAAV. In yet another embodiment, the CD19 inhibitor is administered to the subject at least about 1 month, at least about 2 months, or at least about 3 months before administration of the rAAV.

In one embodiment, the CD19 inhibitor is administered once before administration of the rAAV. In another embodiment, the CD19 inhibitor is administered twice before administration of the rAAV. In yet another embodiment, the CD19 inhibitor is administered 3, 4, 5, or more times before administration of the rAAV.

Administration of the CD19 inhibitor to a human subject can be by any route, including but not limited to intravenous, intradermal, transdermal, subcutaneous, intramuscular, inhalation (e.g., via an aerosol), buccal (e.g., sub-lingual), topical (i.e., both skin and mucosal surfaces, including airway surfaces), intrathecal, intraarticular, intraplural, intracerebral, intra-arterial, intraperitoneal, oral, intralymphatic, intranasal, rectal, or vaginal administration, by perfusion through a regional catheter, or by direct intralesional injection. In one embodiment, a CD19 inhibitor is administered by intravenous push or intravenous infusion given over defined period (e.g., 0.5 to 2 hours). CD19 inhibitors can be delivered by peristaltic means or in the form of a depot, although the most suitable route in any given case will depend, as is well known in the art, on such factors as the species, age, gender, and overall condition of the subject, the nature and severity of the condition being treated, and/or on the nature of the particular composition (i.e., dosage, formulation) that is being administered. In some embodiments, the route of administration is via bolus or continuous infusion over a period of time.

In certain embodiments, the CD19 inhibitor is administered subcutaneously, intramuscularly, intradermally, intraperitoneally, or intravenously. In an exemplary embodiment, the CD19 inhibitor is administered intravenously.

In certain embodiments, the dose of a composition comprising a CD19 inhibitor, e.g. an anti-CD19 antibody, is measured in units of mg/kg of subject body weight. In other embodiments, the dose of a composition comprising a CD19 inhibitor, e.g. an anti-CD19 antibody, is measured in units of mg per dose administered to a subject. Any measurement of dose can be used in conjunction with compositions and methods of the invention and dosage units can be converted by means standard in the art.

In certain embodiments wherein the CD19 inhibitor is an anti-CD19 antibody, the anti-CD19 antibody may be administered at a dose of about 10 mg to about 3000 mg. In some embodiments, the anti-CD19 antibody is be administered at a dose of about 50 mg to about 2000 mg. In some embodiments, the anti-CD19 antibody is be administered at a dose of about 100 mg to about 1000 mg. In some embodiments, the anti-CD19 antibody is be administered at a dose of about 200 mg to about 500 mg. In some embodiments, the anti-CD19 antibody is administered at a dose of about 250 mg to about 350 mg. In an exemplary embodiment, the anti-CD19 antibody is administered at a dose of about 300 mg.

In certain embodiments wherein the CD19 inhibitor is an anti-CD19 antibody, the anti-CD19 antibody may be administered at a dose of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 2000 mg, about 2500 mg, or about 3000 mg.

In certain embodiments wherein the CD19 inhibitor is an anti-CD19 antibody, the anti-CD19 antibody may be administered at a dose of about 0.1 mg/kg to about 50 mg/kg of body weight of a subject. In some embodiments, the anti-CD19 antibody is administered at a dose of about 1 mg/kg to about 10 mg/kg. In some embodiments, the anti-CD19 antibody is administered at a dose of about 2 mg/kg to about 8 mg/kg. In some embodiments, the anti-CD19 antibody is administered at a dose of about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, or about 8 mg/kg.

In certain embodiments wherein the CD19 inhibitor is an anti-CD19 antibody, the anti-CD19 antibody may be administered at a dose of at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 1.5 mg/kg, at least about 2 mg/kg, at least about 2.5 mg/kg, at least about 3 mg/kg, at least about 3.5 mg/kg, at least about 4 mg/kg, at least about 4.5 mg/kg, at least about 5 mg/kg, at least about 5.5 mg/kg, at least about 6 mg/kg, at least about 6.5 mg/kg, at least about 7 mg/kg, at least about 7.5 mg/kg, at least about 8 mg/kg, at least about 8.5 mg/kg, at least about 9 mg/kg, at least about 9.5 mg/kg, at least about 10 mg/kg, at least about 10.5 mg/kg, at least about 11 mg/kg, at least about 11.5 mg/kg, at least about 12 mg/kg, at least about 12.5 mg/kg, at least about 13 mg/kg, at least about 13.5 mg/kg, at least about 14 mg/kg, at least about 14.5 mg/kg, at least about 15 mg/kg, at least about 15.5 mg/kg, at least about 16 mg/kg, at least about 16.5 mg/kg, at least about 17 mg/kg, at least about 17.5 mg/kg, at least about 18 mg/kg, at least about 18.5 mg/kg, at least about 19 mg/kg, at least about 19.5 mg/kg, at least about 20 mg/kg, at least about 25 mg/kg, at least about 30 mg/kg, at least about 35 mg/kg, at least about 40 mg/kg, at least about 45 mg/kg, or at least about 50 mg/kg of body weight of a subject.

In certain embodiments where the CD19 inhibitor is an anti-CD19 antibody, the anti-CD19 antibody may formulated in a pharmaceutical composition that comprises the anti-CD19 antibody and a pharmaceutically acceptable carrier or excipient. In some embodiments, the pharmaceutical composition is formulated for subcutaneous, intramuscular, intradermal, intraperitoneal, or intravenous administration. In an exemplary embodiment, the pharmaceutical composition is formulated for intravenous administration.

In some embodiments, the method for administering a recombinant adeno-associated virus (rAAV) to a subject may further comprise the administration of intravenous immune globulin (IVIg) before, simultaneous with, or after administration of the CD19 inhibitor. In some embodiments, the CD19 inhibitor and the IVIg may be administered simultaneously. In some embodiments, the CD19 inhibitor and the IVIg may be administered sequentially. In some embodiments, the CD19 inhibitor and the IVIg may be administered separately.

In some embodiments, the IVIg may be selected from Bivigam, Clairyg, Flebogam, Flebogammadit, Gammagard Liquid 10%, Gammaplex, Gammunex, IG Vena, Intratecth, Kiovig, Nanogam, Octagam, Octagam 10%, Polyglobin N10%, Sandoglobulin NF liquid, and Vigam. In an exemplary embodiment, the IVIg is Gammagard Liquid 10% (Baxter Healthcare International), which is a sterile, liquid preparation of highly purified and concentrated immunoglobulin G (IgG) antibodies. A non-limiting listing of commercially available immunoglobulin formulations which can be used in methods of the present disclosure as IVIg is provided in U.S. 2017/0021114, the disclosure of which are herein incorporated by reference.

In some embodiments, the method for administering a recombinant adeno-associated virus (rAAV) to a subject may further comprise the administration of a corticosteroid before, simultaneous with, or after administration of the CD19 inhibitor. In some embodiments, the CD19 inhibitor and the corticosteroid may be administered simultaneously. In some embodiments, the CD19 inhibitor and the corticosteroid may be administered sequentially. In some embodiments, the CD19 inhibitor and the corticosteroid may be administered separately.

In some embodiments, the corticosteroid is selected from prednisolone, dexamethasone, hydrocortisone, methylprednisolone, betamethasone, cortisone, prednisone, budesonide, and triamcinolone. In certain exemplary embodiments, the corticosteroid is prednisolone.

In some embodiments, the corticosteroid is administered at a dose of about 10 mg per day to about 100 mg per day. In some embodiments, the corticosteroid is administered at a dose of about 20 mg per day to about 80 mg per day. In some embodiments, the corticosteroid is administered at a dose of about 30 mg per day to about 70 mg per day. In certain exemplary embodiments, the corticosteroid is administered at a dose of about 60 mg per day.

In some embodiments, the corticosteroid is administered at a dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 150 mg, about 175 mg, about 200 mg, about 250 mg, about 300 mg, about 350 mg, about 400 mg, about 450 mg, or about 500 mg.

In some embodiments, the corticosteroid may be administered at a dose of about 0.1 mg/kg/day to about 10 mg/kg/day of body weight of a subject. In some embodiments, the corticosteroid is administered at a dose of about 0.5 mg/kg/day to about 5 mg/kg/day. In some embodiments, the corticosteroid is administered at a dose of about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 2.0, about 2.5, about 3.0, about 3.5, about 4.0, about 4.5, or about 5 mg/kg/day.

In some embodiments, the corticosteroid may be administered for a total of at least 1, 2, 3, 4, 5, 6, 7, 8, 9, 10, or more days prior to administration of the rAAV. For example, in certain exemplary embodiments, the corticosteroid may be administered at 60 mg per day for 5 days prior to administration of the rAAV.

In some embodiments, the corticosteroid may be administered at 60 mg per day for 4 weeks with a first dose occurring 5 days prior to administration of the rAAV. In some embodiments, the corticosteroid may be administered at 60 mg per day for 4 weeks with a first dose occurring 5 days prior to administration of the rAAV, followed by a taper of corticosteroid for an additional 4 weeks.

In some embodiments, the method for administering a recombinant adeno-associated virus (rAAV) to a subject may further comprise the administration of an agent selected from a proteasome inhibitor (e.g., bortezomib, carfilzomib, ixazomib, oprozomib, delanzomib, or marizomib), a toll-like receptor (TLR) antagonist (e.g., a TLR7 antagonist or a TLR9 antagonist such as chloroquine, hydroxychloroquine, or quinacrine, AT791 or E6446 (see Lamphier et al., 2014, Mol Pharmacol., 85 (3): 429-40)), an immunosuppressive macrocycle (e.g., sirolimus, cyclosporin A, tacrolimus, or rapamycin analogues such as everolimus, biolimus, and temsirolimus), an antimetabolite (e.g., azathioprine, mercaptopurine, thiopurine, or methotrexate), a cyclic guanosine monophosphate (GMP)-adenosine monophosphate (AMP) synthase-stimulator of interferon genes (cGAS-STING) antagonist, interleukin-2 (IL-2), an IL-2 mutein (e.g., AMG 592, NKTR-358, or DEL106), an anti-CD38 antibody (e.g., daratumumab, isatuximab, MOR202, or TAK-079), an immunomodulatory drug (IMID) (e.g., lenalidomide, pomalidomide, or apremilast), an anti-B-cell maturation antigen (BCMA) agent (e.g., chimeric antigen receptor T-cells (CAR-T) (e.g., CT053 or bb2121), antibody-drug conjugate (ADC) (e.g., GSK2857916), bi-specific T cell engager (BiTE), or an anti-BCMA antibody), an anti-SLAM family member 7 (SLAMF7) antibody (e.g., elotuzumab), a mammalian target of rapamycin (mTOR) inhibitor (e.g., ABI-009 nab-rapamycin), synthetic vaccine particles encapsulating rapamycin (SVP-R), the combination of SVP-R and pegylated uricase (pegadricase) (e.g., SEL-212), cyclophosphamide, mycophenolate mofetil, a phosphoinositide 3-kinase inhibitor (e.g., idelalisib, copanlisib, duvelisib, or alpelisib), a Bruton's tyrosine kinase inhibitor (e.g., ibrutinib, zanubrutinib, acalabrutinib, evobrutinib, HM71224, BGB-3111, spebrutinib, or ONO-4059), a sphingosine-1-phosphate receptor modulator (e.g., fingolimod or ozanimod), an anti-B-cell activating factor (BAFF or also known as tumor necrosis factor ligand superfamily member 13B) inhibitor (e.g., belimumab, atacicept, or blisibimod), or an IgG-degrading protease (e.g., IdeS of Streptococcus pyogenes, an engineered IdeS variant, IdeZ of Streptoccus equi, or IgdE enzymes from Streptococcus suis, Streptococcus porcinus, or Streptococcus equi). In some embodiments, the agent may be administered before, simultaneous with, or after administration of the CD19 inhibitor. In some embodiments, the CD19 inhibitor and the agent may be administered simultaneously. In some embodiments, the CD19 inhibitor and the agent may be administered sequentially. In some embodiments, the CD19 inhibitor and the agent may be administered separately.

In some embodiments, the agent is a proteasome inhibitor. In some embodiments, the proteasome inhibitor is selected from bortezomib, carfilzomib, ixazomib, oprozomib, delanzomib, or marizomib. In an exemplary embodiment, the proteasome inhibitor is bortezomib. Accordingly, in some embodiments, this disclosure provides a method for administering a recombinant adeno-associated virus (rAAV) to a subject, wherein said method comprises first administering a CD19 inhibitor and a proteasome inhibitor to said subject and thereafter administering the rAAV to said subject. In some embodiments, the CD19 inhibitor is inebilizumab and the proteasome inhibitor is bortezomib. Thus, in some embodiments, the instant disclosure provides a method of administering an rAAV following administration of an anti-CD19 antibody such as inebilizumab and a proteasome inhibitor such as bortezomib.

In some embodiments, the agent is an IgG-degrading protease. Examples of proteases that may be used in the instant invention include, for example and without limitation, those described in WO/2020/016318 and/or WO/2020/159970, including, for example, cysteine proteases from Streptococcus pyogenes, Streptococcus equi, Mycoplasma canis, Streptococcus agalactiae, Streptococcus pseudoporcinus, or Pseudomonas putida.

In certain embodiments, the IgG-degrading protease is the IdeS from Streptococcus pyogenes (SEQ ID NO: 20) or a protease which is at least 80%, at least 85%, at least 90%, at least 95%, at least 96%, at least 97%, at least 98%, or at least 99% identical to SEQ ID NO: 20. In some embodiments, the protease is an engineered variant of SEQ ID NO: 20. Examples of engineered IdeS proteases are described in WO/2020/016318 and U.S. Patent Publication Nos. 20180023070 and 20180037962. In some embodiments, the engineered IdeS variant may have 1, 2, 3, 4, 5, or more amino acid modifications relative to SEQ ID NO: 20.

In certain embodiments, the IgG-degrading protease is the IdeZ from Streptococcus equi (SEQ ID NO: 21) or a protease which is at least 80%, at least 85%, at least 90%, at least 95%, at least 96%, at least 97%, at least 98%, or at least 99% identical to SEQ ID NO: 21. In some embodiments, the protease is an engineered variant of SEQ ID NO: 21. Examples of engineered IdeZ proteases are described in WO/2020/016318. In some embodiments, the engineered IdeZ variant may have 1, 2, 3, 4, 5, or more amino acid modifications relative to SEQ ID NO: 21.

Other proteases that may be used in the instant invention include, for example and without limitation, IgdE enzymes from Streptococcus suis, Streptococcus porcinus, and Streptococcus equi, described in WO/2017/134274.

In some embodiments, the IgG-degrading protease may be encapsulated in or complexed with liposomes, nanoparticles, lipid nanoparticles (LNPs), polymers, microparticles, microcapsules, micelles, or extracellular vesicles.

In some embodiments, the recombinant adeno-associated virus (rAAV) administered according to a method of the present disclosure comprises an AAV capsid and a vector genome packaged therein.

In some embodiments, the packaged vector genome comprises an AAV 5′ inverted terminal repeat sequence (ITR), a promoter sequence, a partial or complete coding sequence for a protein transgene or a functional fragment, functional variant or isoform thereof, and an AAV 3′ inverted terminal repeat sequence (ITR).

In some embodiments, the packaged genome may further comprise an enhancer, an intron, a consensus Kozak sequence, and/or a polyadenylation signal. In some embodiments, the recombinant vector can further include one or more stuffer nucleic acid sequences. In one embodiment, a stuffer nucleic acid sequence is situated between the intron and the partial or complete coding sequence for a protein transgene.

In various embodiments described herein, the rAAV comprises an AAV capsid. AAV capsids of any serotype may be used in the present invention, and the selection of AAV serotype will depend in part on the cell type(s) that are targeted for gene therapy. The AAV capsid can be from an AAV of serotype 1, 2, 3, 4, 5, 6, 7, 8, 9, 10, 11, 12, rh10, hu37 (i.e., AAV1, AAV2, AAV3, AAV4, AAV5, AAV6, AAV7, AAV8, AAV9, AAV10, AAV11, AAV12, AAVrh10, AAVhu37), as well as any one of the more than 100 variants isolated from human and nonhuman primate tissues (See, e.g., Choi et al., 2005, Curr Gene Ther. 5:299-310, 2005 and Gao et al., 2005, Curr Gene Ther. 5:285-297).

Beyond the aforementioned capsids, also included within the scope of the present disclosure are variant AAV capsids which have been engineered to harbor one or more beneficial therapeutic properties (e.g., improved targeting for select tissues, increased ability to evade the immune response, reduced stimulation of neutralizing antibodies, etc.). Non-limiting examples of such engineered variant capsids are described in U.S. Pat. Nos. 9,506,083, 9,585,971, 9,587,282, 9,611,302, 9,725,485, 9,856,539, 9,909,142, 9,920,097, 10,011,640, 10,081,659, 10,179,176, 10,202,657, 10,214,566, 10,214,785, 10,266,845, 10,294,281, 10,301,648, 10,385,320, and 10,392,632 and in PCT Publication Nos. WO/2017/165859, WO/2018/022905, WO/2018/156654, WO/2018/222503, and WO/2018/226602, the disclosures of which are herein incorporated by reference.

In certain exemplary embodiments, the rAAV administered according to the present disclosure comprises an AAV9 capsid. The AAV9 capsid is a self-assembled AAV capsid composed of multiple AAV9 vp proteins. The AAV9 vp proteins are typically expressed as alternative splice variants encoded by a nucleic acid sequence of SEQ ID NO: 18 or a sequence that is at least 70%, at least 75%, at least 80%, at least 85%, at least 90%, at least 95%, at least 97%, at least 99% thereto, which encodes the vp1 amino acid sequence of SEQ ID NO: 19 (GenBank Accession: AAS99264). These splice variants result in proteins of different length of SEQ ID NO: 19. In certain embodiments, an AAV9 capsid includes an AAV having an amino acid sequence which is 99% identical to AAS99264 or 99% identical to SEQ ID NO: 19. See also U.S. Pat. No. 7,906,111, and WO/2005/033321. As used herein, an AAV9 variant includes those described in, e.g., WO/2016/049230, U.S. Pat. No. 8,927,514, U.S. Patent Publication No. 2015/0344911, and U.S. Pat. No. 8,734,809.

As indicated herein, the rAAV administered according to the invention may comprise, in some embodiments, an AAV9 capsid. However, in other embodiments, another AAV capsid is selected. Tissue specificity is determined by the capsid type. AAV serotypes which transduce a suitable target (e.g., liver, muscle, lung, or CNS) may be selected as sources for capsids of AAV viral vectors including, e.g., AAV1, AAV2, AAV3, AAV4, AAV5, AAV6, AAV6.2, AAV7, AAV8, AAV9, AAVrh10, AAVrh64R1, AAVrh64R2, AAVrh8. See, e.g., U.S. Patent Publication No 2007/0036760; U.S. Patent Publication No. 2009/0197338; and EP1310571. See also WO 2003/042397 (AAV7 and other simian AAV), U.S. Pat. Nos. 7,282,199 and 7,790,449 (AAV8). In addition, AAV yet to be discovered, or a recombinant AAV based thereon, may be used as a source for the AAV capsid. These documents also describe other AAV which may be selected for generating AAV and are incorporated by reference. In some embodiments, an AAV capsid for use in the viral vector can be generated by mutagenesis (i.e., by insertions, deletions, or substitutions) of one of the aforementioned AAV capsids or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned capsids

In some embodiments, the rAAV comprises a packaged vector genome which comprises an AAV ITR sequence, which functions as both the origin of vector DNA replication and the packaging signal of the vector genome, when AAV and adenovirus helper functions are provided in trans. Additionally, the ITRs serve as the target for single-stranded endonucleatic nicking by the large Rep proteins, resolving individual genomes from replication intermediates.

In some embodiments, the 5′-ITR sequence is from AAV2. In some embodiments, the 3′-ITR sequence is from AAV2. In some embodiments, the 5′-ITR sequence and the 3′-ITR sequence are from AAV2. In some embodiments, the 5′-ITR sequence and/or the 3′-ITR sequence are from AAV2 and comprise or consist of SEQ ID NO: 17. In other embodiments, the 5′-ITR sequence and/or the 3′-ITR sequence are from a non-AAV2 source.

In various aspects described herein, rAAV used in the methods disclosed herein may contain a packaged genome which comprises a promoter sequence which helps drive and regulate transgene expression, e.g., expression of a protein transgene. In exemplary embodiments, the promoter sequence is located between the selected 5′ ITR sequence and the partial or complete coding sequence for the protein transgene. In some embodiments, the promoter sequence is located downstream of an enhancer sequence. In some embodiments, the promoter sequence is located upstream of an intron sequence.

In some embodiments, the promoter is selected from a chicken β-actin (CBA) promoter, a cytomegalovirus immediate early gene (CMV) promoter, a transthyretin (TTR) promoter, a thyroxine binding globulin (TBG) promoter, an alpha-1 anti-trypsin (A1AT) promoter, and a CAG promoter.

In addition to a promoter, a vector may contain other appropriate transcription initiation, termination, enhancer sequence, and efficient RNA processing signals. Such sequences include splicing and polyadenylation (poly A) signals, regulatory elements that enhance expression (i.e., WPRE), sequences that stabilize cytoplasmic mRNA, sequences that enhance translation efficiency (i.e., the Kozak consensus sequence), and sequences that enhance protein stability.

In various aspects described herein, rAAV used in the methods disclosed herein may contain a packaged genome that comprises an AAV 5′ inverted terminal repeat sequence (ITR), a promoter sequence, a partial or complete coding sequence for a protein transgene or a functional fragment, functional variant or isoform thereof, and an AAV 3′ inverted terminal repeat sequence (ITR).

In one embodiment, the partial or complete coding sequence for the protein transgene is a wild-type coding sequence. As used herein, the term “wild-type” refers to a biopolymer (e.g., a polypeptide sequence or polynucleotide sequence) that is the same as the biopolymer (e.g., polypeptide sequence or polynucleotide sequence) that exists in nature.

In an alternative embodiment, the partial or complete coding sequence for the protein transgene is a codon-optimized coding sequence. In one embodiment, the partial or complete coding sequence for the protein transgene is codon-optimized for expression in humans.

In various embodiments described herein, rAAV are provided that contain a packaged genome that comprises a coding sequence for a protein transgene. The polypeptides delivered with the rAAV described herein encompass polypeptides that may be useful in the treatment of mammals, including humans. Such polypeptides include, but are not limited to, ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit α (PCCA) and propionyl-CoA carboxylase subunit β (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, and a dystrophin gene product (e.g., a minidystrophin or microdystrophin). A non-limiting list of suitable transgenes is found in WO/2019/168961, the disclosure of which is herein incorporated by reference.

In some embodiments, the invention may be used to deliver fragments of the aforementioned transgene proteins which comprise at least 50, at least 100, at least 150, at least 200, at least 250, at least 300, at least 350, at least 400, at least 450, at least 500, at least 550, or at least 600 amino acid residues and retain one or more activities associated with the full-length polypeptide (e.g., catalytic activity in the case of an enzyme). Such fragments may be obtained by recombinant techniques that are routine and well-known in the art. Moreover, such fragments may be tested for catalytic activity by routine in vitro assays known to the skilled artisan.

In some embodiments, the invention may be used to deliver variants of the aforementioned transgene proteins. In some embodiments, the variant proteins may be at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100%) identical to the wild-type therapeutic protein. In some embodiments, the variant transgene proteins may have at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 different residues as compared to the respective wild-type protein. Such variants may be obtained by recombinant techniques that are routine and well-known in the art. Moreover, such variants may be tested for catalytic activity by routine in vitro assays known to the skilled artisan.

In some embodiments, the rAAV to be administered according to the methods of the present disclosure may be formulated as a pharmaceutical composition that comprises the rAAV and a pharmaceutically acceptable carrier or excipient. Suitable pharmaceutical formulations for administration of rAAV can be found, for example, in U.S. Patent Application Publication No. 2012/0219528. The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents. In some embodiments, the pharmaceutical composition comprising an rAAV is formulated for subcutaneous, intramuscular, intradermal, intraperitoneal, intrathecal, intracerebroventricular, or intravenous administration. In an exemplary embodiment, the pharmaceutical composition is formulated for intravenous administration.

In some embodiments, the rAAV is formulated in a buffer/carrier suitable for infusion in human subjects. The buffer/carrier should include a component that prevents the rAAV from sticking to the infusion tubing but does not interfere with the rAAV binding activity in vivo. Various suitable solutions may include one or more of: a buffering saline, a surfactant, and a physiologically compatible salt or mixture of salts adjusted to an ionic strength equivalent to about 100 mM sodium chloride (NaCl) to about 250 mM sodium chloride, or a physiologically 20) compatible salt adjusted to an equivalent ionic concentration. The pH may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene 10 (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol.

In some embodiments, viral particles (e.g., recombinant AAV) may be encapsulated in or complexed with liposomes, nanoparticles, lipid nanoparticles (LNPs), polymers, microparticles, microcapsules, micelles, or extracellular vesicles.

In yet another aspect, the disclosure provides a method for administering a nucleic acid (e.g., DNA, siRNA, or mRNA) to a subject, wherein said method comprises first administering a CD19 inhibitor to said subject and thereafter administering the nucleic acid (e.g., DNA, siRNA, or mRNA) to said subject. In an exemplary embodiment, the nucleic acid is an mRNA encoding a protein. Any therapeutic protein of interest (e.g., a human wild-type protein) is suitable for use in the methods described herein. An exemplary listing of human proteins can be found in WO/2020/118239. In specific embodiments, the protein is selected from ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit α (PCCA) and propionyl-CoA carboxylase subunit β (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, and a dystrophin gene product (e.g., a minidystrophin or microdystrophin).

In certain embodiments, the nucleic acid (e.g., DNA, siRNA, or mRNA) is encapsulated in or complexed with a liposome, nanoparticle, lipid nanoparticle (LNP), polymer, microparticle, microcapsule, micelle, or extracellular vesicle.

In some embodiments, the nucleic acid (e.g., DNA, siRNA, or mRNA) is encapsulated in a lipid nanoparticle (LNP). Examples of LNPs capable of delivering a nucleic acid are described in International Patent Publication Nos. WO/2015/074085, WO/2016/081029, WO/2017/117530, WO/2018/118102, WO/2018/119163, WO/2018/222926, WO/2019/191780, WO/2020/016318, and WO/2020/154746.

In one embodiment, a lipid nanoparticle for use in the instant invention comprises (a) a nucleic acid (e.g., DNA, siRNA, or mRNA), (b) a cationic lipid, (c) an aggregation reducing agent (such as a PEG-lipid), (d) optionally a non-cationic lipid (such as a neutral lipid), and (e) optionally a sterol. In one embodiment, the lipid nanoparticle comprises (i) at least one cationic lipid; (ii) a neutral lipid, e.g., DSPC; (iii) a sterol, e.g., cholesterol; and (iv) a PEG-lipid, in a molar ratio of about 20-65% cationic lipid: 5-25% neutral lipid: 25-55% sterol; 0.5-15% PEG-lipid. In some embodiments, the cationic lipid is selected from ATX-002, ATX-081, ATX-095, or ATX-126, as described in WO/2018/222926.

In yet another aspect, the disclosure provides a method for treating a disorder in a subject, wherein said method comprises first administering a CD19 inhibitor to said subject and thereafter administering a therapeutically effective amount of a recombinant adeno-associated virus (rAAV) to said subject. As used herein, “disease,” “disorder” and “condition” are used interchangeably, to indicate an abnormal state in a subject.

As will be appreciated by the skilled artisan equipped with the present disclosure, individuals suffering from one or more of a variety of disorders may benefit from a method of therapeutic recombinant AAV administration described herein. In some embodiments, the disorder may be selected from OTC deficiency, glycogen storage disease type 1a (GSD1a), hemophilia A, hemophilia B, Wilson disease, phenylketonuria, citrullinemia, CDKL5 deficiency (CDD), propionic acidemia, spinal muscular atrophy, mucopolysaccharidosis type I, mucopolysaccharidosis type II, CLN2 disease, homozygous familiar hypercholesterolemia, Pompe disease, X-linked myotubular myopathy, myotonic dystrophy, Duchenne muscular dystrophy, cystic fibrosis, methylmalonic acidemia, Parkinson's disease, Alzheimer's disease, and Huntington's disease.

Any suitable method or route can be used to administer a rAAV or a rAAV-containing composition described herein. Routes of administration include, for example, systemic, oral, inhalation, intranasal, intratracheal, intraarterial, intraocular, intravenous, intrathecal, intracerebroventricular, intramuscular, subcutaneous, intradermal, and other parental routes of administration. In some embodiments, the rAAV, or a composition comprising a rAAV, is administered intravenously.

The specific dose administered can be a uniform dose for each subject, for example, 1.0×10¹¹-1.0×10¹⁴ genome copies (GC) of virus per subject. Alternatively, a subject's dose can be tailored to the approximate body weight or surface area of the subject. Other factors in determining the appropriate dosage can include the disease or condition to be treated or prevented, the severity of the disease, the route of administration, and the age, sex and medical condition of the subject. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual subject's dosage can also be adjusted as the progress of the disease is monitored.

In some embodiments, the rAAV is administered at a dose of, e.g., about 1.0×10¹¹ genome copies per kilogram of subject body weight (GC/kg) to about 1×10¹⁴ GC/kg, about 5×10¹¹ genome copies per kilogram of subject body weight (GC/kg) to about 5×10¹³ GC/kg, or about 1×10¹² to about 1×10¹³ GC/kg, as measured by qPCR or digital droplet PCR (ddPCR). In some embodiments, the rAAV is administered at a dose of about 2×10¹² GC/kg. In some embodiments, the rAAV is administered at a dose of about 6×10¹² GC/kg. In some embodiments, the rAAV is administered at a dose of about 1×10¹³ GC/kg.

In a further aspect, the present disclosure provides a method for administering at least two doses of recombinant adeno-associated virus (rAAV) to a subject, wherein said method comprises:
(a) administering a first dose of a CD19 inhibitor to said subject and thereafter administering a first rAAV to said subject, and
(b) administering a second dose of a CD19 inhibitor to said subject and thereafter administering a second rAAV to said subject.

In some embodiments according to this second aspect, the CD19 inhibitor of the first and second doses is the same CD19 inhibitor. Alternatively, the CD19 inhibitor of the first dose may be a different CD19 inhibitor relative to the second dose.

In some embodiments according to this second aspect, the first rAAV and the second rAAV are the same rAAV. Alternatively, the first rAAV and the second rAAV are different.

In some embodiments according to this second aspect, the first rAAV and the second rAAV contain the same vector capsid. In an alternative embodiment, the first rAAV and the second rAAV contain different vector capsids.

In some embodiments according to this second aspect, the first rAAV and the second rAAV express the same transgene. In an alternative embodiment, the first rAAV and the second rAAV express different transgenes.

In some embodiments according to this second aspect, administration of the first dose of the CD19 inhibitor occurs more than 12 months prior to administration of the second dose of the CD19 inhibitor. In one embodiment, administration of the first dose of the CD19 inhibitor occurs more than 2 years prior to administration of the second dose of the CD19 inhibitor. In another embodiment, administration of the first dose of the CD19 inhibitor occurs more than 3, more than 4, more than 5, more than 6, more than 7, more than 8, more than 9, more than 10, more than 15, or more than 20 years prior to administration of the second dose of the CD19 inhibitor.

In some embodiments according to this second aspect, the CD19 inhibitor of the first dose is an anti-CD19 antibody. In some embodiments according to this second aspect, the CD19 inhibitor of the second dose is an anti-CD19 antibody. In some embodiments according to this second aspect, the CD19 inhibitor of the first and second doses is an anti-CD19 antibody. In one embodiment, the anti-CD19 antibody is selected from inebilizumab, tafasitamab, and FMC63. In an exemplary embodiment, the anti-CD19 antibody is inebilizumab.

In some embodiments according to this second aspect, the amount of CD19 inhibitor administered in the first and second doses is the same. Alternatively, the amount of CD19 inhibitor administered in the first dose may be different than the amount of the CD19 inhibitor administered in the second dose. In an exemplary embodiment, the amount of CD19 inhibitor administered in the first and second doses is the same. In another exemplary embodiment, the CD19 inhibitor is inebilizumab and the first dose is 300 mg and the second dose is 300 mg.

In some embodiments according to this second aspect, the regimen of dosing a CD19 inhibitor followed by administration of an rAAV may be repeated 3, 4, 5, or more times. Accordingly, in some embodiments according to this second aspect, the method may further comprise step (c) of administering a third dose of a CD19 inhibitor to said subject and thereafter administering a third rAAV to said subject.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

What is claimed is:

1. A method for administering a recombinant adeno-associated virus (rAAV) to a subject, wherein said method comprises first administering a CD19 inhibitor to said subject and thereafter administering the rAAV to said subject.

2. The method of claim 1, wherein the CD19 inhibitor is administered to the subject at least about 12 hours before administration of the rAAV.

3. The method of claim 1, wherein the CD19 inhibitor is administered to the subject at least about 24 hours before administration of the rAAV.

4. The method of claim 1, wherein the CD19 inhibitor is administered to the subject at or about 7 days before administration of the rAAV.

5. The method of claim 1, wherein the CD19 inhibitor is administered to the subject at or about 14 days before administration of the rAAV.

6. The method of claim 1, wherein the CD19 inhibitor is administered to the subject at or about 21 days before administration of the rAAV.

7. The method of claim 1, wherein the CD19 inhibitor is administered once before administration of the rAAV.

8. The method of claim 1, wherein the CD19 inhibitor is administered twice before administration of the rAAV.

9. The method of any of the preceding claims, wherein the CD19 inhibitor is selected from an anti-CD19 antibody, an anti-CD19 scFv, a CD19 antisense oligonucleotide, a CD19 siRNA, and a small molecule inhibitor of CD19.

10. The method of claim 9, wherein the CD19 inhibitor is an anti-CD19 antibody.

11. The method of claim 10, wherein the anti-CD19 antibody is selected from inebilizumab, tafasitamab, and FMC63.

12. The method of claim 11, wherein the anti-CD19 antibody is inebilizumab.

13. The method of claim 10, wherein the anti-CD19 antibody is administered at a dose of about 10 mg to about 3000 mg.

14. The method of claim 10, wherein the anti-CD19 antibody is administered at a dose of about 50 mg to about 2000 mg.

15. The method of claim 10, wherein the anti-CD19 antibody is administered at a dose of about 100 mg to about 1000 mg.

16. The method of claim 10, wherein the anti-CD19 antibody is administered at a dose of about 200 mg to about 500 mg.

17. The method of claim 10, wherein the anti-CD19 antibody is administered at a dose of about 300 mg.

18. The method of claim 10, wherein the anti-CD19 antibody is administered at a dose of about 1 mg/kg to about 10 mg/kg.

19. The method of claim 10, wherein the anti-CD19 is administered at a dose of about 2 mg/kg to about 8 mg/kg.

20. The method of claim 10, wherein the CD19 antibody is formulated in a pharmaceutical composition that comprises the anti-CD19 antibody and a pharmaceutically acceptable carrier or excipient.

21. The method of claim 20, wherein the pharmaceutical composition is formulated for subcutaneous, intramuscular, intradermal, intraperitoneal, or intravenous administration.

22. The method of claim 21, wherein the pharmaceutical composition is formulated for intravenous administration.

23. The method of claim 1, wherein the CD19 inhibitor is administered subcutaneously, intramuscularly, intradermally, intraperitoneally, or intravenously.

24. The method of claim 23, wherein the CD19 inhibitor is administered intravenously.

25. The method of any of the preceding claims, wherein the method further comprises administering intravenous immune globulin (IVIg) before, simultaneous with, or after administration of the CD19 inhibitor.

26. The method of any of the preceding claims, wherein the method further comprises the administration of a corticosteroid before, simultaneous with, or after administration of the CD19 inhibitor.

27. The method of claim 26, wherein the corticosteroid is selected from prednisolone, dexamethasone, hydrocortisone, methylprednisolone, betamethasone, cortisone, prednisone, budesonide, and triamcinolone.

28. The method of claim 27, wherein the corticosteroid is prednisolone and is administered at a dose of 60 mg per day.

29. The method of claim 27, wherein prednisolone is administered at about 5 days prior to administration of the rAAV.

30. The method of any of the preceding claims, wherein the method further comprises the administration of an agent selected from a proteasome inhibitor, a TLR antagonist, an immunosuppressive macrocycle, an antimetabolite, a cGAS-STING antagonist, IL-2, an IL-2 mutein, an anti-CD38 antibody, an immunomodulatory drug (IMiD), an anti-B-cell maturation antigen (BCMA) agent, an anti-SLAM family member 7 (SLAMF7) antibody, a mammalian target of rapamycin (mTOR) inhibitor, SEL-212, cyclophosphamide, mycophenolate mofetil, a phosphoinositide 3-kinase inhibitor, a Bruton's tyrosine kinase inhibitor, a sphingosine-1-phosphate receptor modulator, an anti-B-cell activating factor (BAFF or also known as tumor necrosis factor ligand superfamily member 13B) inhibitor, or an IgG-degrading protease before, simultaneous with, or after administration of the CD19 inhibitor.

31. The method of claim 30, wherein the proteasome inhibitor is selected from bortezomib, carfilzomib, ixazomib, oprozomib, delanzomib, and marizomib.

32. The method of claim 31, wherein the proteasome inhibitor is bortezomib.

33. The method of claim 30, wherein the IgG-degrading protease is IdeS of Streptococcus pyogenes or an engineered variant thereof.

34. The method of claim 30, wherein the IgG-degrading protease is IdeZ of Streptococcus equi or an engineered variant thereof.

35. The method of any of the preceding claims, wherein the rAAV comprises an AAV capsid and a vector genome packaged therein.

36. The method of claim 35, wherein the AAV capsid is from an AAV of serotype 1, 2, 3, 4, 5, 6, 7, 8, 9, 10, 11, 12, rh10, hu37, or an engineered variant thereof.

37. The method of claim 36, wherein the AAV capsid is an AAV9 capsid.

38. The method of claim 36, wherein the AAV capsid is an AAV8 capsid.

39. The method of claim 36, wherein the AAV capsid is an hu37 capsid.

40. The method of claim 35, wherein the vector genome comprises a coding sequence for a protein transgene.

41. The method of claim 40, wherein the coding sequence is a native coding sequence.

42. The method of claim 40, wherein the coding sequence is a codon-optimized coding sequence.

43. The method of claim 40, wherein the coding sequence expresses a protein transgene selected from ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit α (PCCA) and propionyl-CoA carboxylase subunit β (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, and a dystrophin gene product.

44. The method of any of the preceding claims, wherein the rAAV is administered subcutaneously, intramuscularly, intradermally, intraperitoneally, intrathecally, intracerebroventricularly, or intravenously.

45. The method of claim 44, wherein the rAAV is administered intravenously.

46. The method of any of the preceding claims, wherein the rAAV is administered at a dose of about 1×10¹¹ to about 1×10¹⁴ genome copies (GC)/kg.

47. The method of any of the preceding claims, wherein the subject is a human.

48. The method of claim 47, wherein the human subject is an adult subject.

49. The method of claim 47, wherein the human subject is a pediatric subject.

50. A method for administering at least two doses of recombinant adeno-associated virus (rAAV) to a subject, wherein said method comprises:
(a) administering a first dose of a CD19 inhibitor to said subject and thereafter administering a first rAAV to said subject, and
(b) administering a second dose of a CD19 inhibitor to said subject and thereafter administering a second rAAV to said subject.

51. The method of claim 50, wherein the CD19 inhibitor of the first and second doses is the same CD19 inhibitor.

52. The method of claim 50, wherein the CD19 inhibitor of the first dose is a different CD19 inhibitor relative to the second dose.

53. The method of claim 50, wherein the first rAAV and the second rAAV are the same rAAV.

54. The method of claim 50, wherein the first rAAV and the second rAAV are different rAAVs.

55. The method of claim 50, wherein the first rAAV and the second rAAV contain the same AAV capsid.

56. The method of claim 50, wherein the first rAAV and the second rAAV contain different AAV capsids.

57. The method of claim 50, wherein the first rAAV and the second rAAV express the same transgene.

58. The method of claim 50, wherein the first rAAV and the second rAAV express different transgenes.

59. The method of claim 50, wherein administration of the first dose of the CD19 inhibitor occurs more than 12 months prior to administration of the second dose of the CD19 inhibitor.

60. The method of claim 50, wherein administration of the first dose of the CD19 inhibitor occurs more than 2 years prior to administration of the second dose of the CD19 inhibitor.

61. The method of claim 50, wherein administration of the first dose of the CD19 inhibitor occurs more than 10 years prior to administration of the second dose of the CD19 inhibitor.

62. The method of any of claims 50-61, wherein the CD19 inhibitor of the first dose is an anti-CD19 antibody.

63. The method of any of claim 50-61, wherein the CD19 inhibitor of the second dose is an anti-CD19 antibody.

64. The method of any of claims 50-61, wherein the CD19 inhibitor of the first and second doses is an anti-CD19 antibody.

65. The method of any of claims 62-64, wherein the anti-CD19 antibody is selected from inebilizumab, tafasitamab, and FMC63.

66. The method of claim 65, wherein the anti-CD19 antibody is inebilizumab.

67. The method of claim 50, wherein the amount of CD19 inhibitor administered in the first and second doses is the same.

68. The method of claim 50, wherein the amount of the CD19 inhibitor administered in the first and second doses is different.
